(19) United States
(12) Patent Application Publication
(10) Pub. No.: US 2024/0270799 A1
(43) Pub. Date: Aug. 15, 2024

(54) MODIFIED PHOTORECEPTIVE CHLORIDE CHANNEL

(71) Applicant: NATIONAL UNIVERSITY CORPORATION, IWATE UNIVERSITY, Morioka-shi, Iwate (JP)

(72) Inventors: Hiroshi TOMITA, Morioka-shi (JP); Eriko SUGANO, Morioka-shi (JP)

(73) Assignee: NATIONAL UNIVERSITY CORPORATION, IWATE UNIVERSITY, Morioka-shi, Iwate (JP)

(21) Appl. No.: 17/913,769

(22) PCT Filed: Mar. 24, 2021
(86) PCT No.: PCT/JP2021/012283

§ 371 Date: Apr. 23, 2024

(30) Foreign Application Priority Data
Mar. 24, 2020 (JP) 2020-053474

(51) Int. Cl. C07K 14/405; A61K 38/00; A61P 27/02; C12N 15/86

(52) U.S. Cl. CPC C07K 14/405; A61P 27/02; C12N 15/86; A61K 38/00; C07K 2319/03; C12N 2750/14143

(57) ABSTRACT

An object of the present invention is to provide a modified photoreceptive chloride channel having excellent photoreactive properties in which the wavelength sensitivity range is narrow, and also both τon and τoff are short. The solution is a polypeptide obtained by substituting a region from the fourth transmembrane domain to the sixth transmembrane domain counted from the N-terminal side of a Guillardia theta-derived photoreceptive chloride channel-1 (GtACR1) by a corresponding region of a Guillardia theta-derived photoreceptive chloride channel-2 (GtACR2).

### TECHNICAL FIELD

The present invention relates to a modified photoreceptive chloride channel. More specifically, it relates to a modified photoreceptive chloride channel having excellent photoreactive properties in which the wavelength sensitivity range is narrow, and also both a time until the channel opens from the start of light irradiation (opening speed: τon) and a time until the channel closes from the stop of light irradiation (closing speed: τoff) are short.

### BACKGROUND ART

It is well known that a research aiming at reconstruction of visual function by optogenetics to control cellular response by applying light to neurons made to express a photosensitive protein (channelrhodopsin) by gene transfer has been globally performed, and various studies have been conducted so far. As a light-gated channel, a cation channel, which is responsible for the distribution of cations inside and outside cells, and an anion channel, which is responsible for the distribution of anions inside and outside cells, are known. However, it is not long since the light-gated anion channel was discovered, and there are few reports on the modified light-gated anion channel compared to reports on the modified light-gated cation channel. Under such circumstances, the research group of Kato et al. focused on a photoreceptive chloride channel-1 (GtACR1) (Non-Patent Document 1) isolated from Guillardia theta, which is one of green algae, and has reported that a modified photoreceptive chloride channel (FLASH) in which Arg83 and Asn239 are substituted by Glu shows shorter τoff than GtACR1 (Non-Patent Document 2). However, there have been no reports so far on a modified photoreceptive chloride channel having excellent photoreactive properties in which the wavelength sensitivity range is narrow, and also both τon and τoff are short.

### PRIOR ART DOCUMENTS

### Non-Patent Document

Non-Patent Document 1: Govorunova, E. G. et al., Natural light-gated anion channels: a family of microbial rhodopsins for advanced optogenetics., Science, 349, 647-650 (2015)
Non-Patent Document 2: Hideaki E. Kato et al., Structural mechanisms of selectivity and gating in anion channelrhodopsins., Nature, 561, 349-354 (2018)

### SUMMARY OF THE INVENTION

### Problems that the Invention is to Solve

Therefore, an object of the present invention is to provide a modified photoreceptive chloride channel having excellent photoreactive properties in which the wavelength sensitivity range is narrow, and also both τon and τoff are short.

### Means for Solving the Problems

As a result of intensive studies in view of the above points, the present inventors have found that by substituting a region from the fourth transmembrane domain to the sixth transmembrane domain counted from the N-terminal side of GtACR1, which is a 7-transmembrane protein, by a corresponding region (that is, a region from the fourth transmembrane domain to the sixth transmembrane domain counted from the N-terminal side) of a photoreceptive chloride channel-2 (GtACR2), which is a 7-transmembrane protein in the same manner as GtACR1, isolated from Guillardia theta together with GtACR1, not only the wavelength sensitivity range can be made narrower than that of GtACR1 and GtACR2, but also both τon and τoff can be made shorter than those of GtACR1 and GtACR2.

A modified photoreceptive chloride channel of the present invention achieved based on the above-mentioned findings is, as described in claim 1, a polypeptide obtained by substituting a region from the fourth transmembrane domain to the sixth transmembrane domain counted from the N-terminal side of GtACR1 by a corresponding region of GtACR2.

Further, a modified photoreceptive chloride channel described in claim 2 is the modified photoreceptive chloride channel described in claim 1, which is obtained by further substituting an intracellular domain between the third transmembrane domain and the fourth transmembrane domain counted from the N-terminal side of GtACR1, and/or an extracellular domain between the sixth transmembrane domain and the seventh transmembrane domain counted therefrom by a corresponding domain of GtACR2.

Further, a modified photoreceptive chloride channel described in claim 3 is the modified photoreceptive chloride channel described in claim 1 or 2, which is any of the following (a) to (c):
(a) a polypeptide composed of the amino acid sequence represented by SEQ ID NO: 3;
(b) a polypeptide that is composed of an amino acid sequence including deletion, substitution, addition, or insertion of one or a plurality of amino acids in the amino acid sequence represented by SEQ ID NO: 3, and that has a photoreceptive chloride channel function; and
(c) a polypeptide that is composed of an amino acid sequence having at least 90% sequence identity to the amino acid sequence represented by SEQ ID NO: 3, and that has a photoreceptive chloride channel function.

Further, a modified photoreceptive chloride channel described in claim 4 is the modified photoreceptive chloride channel described in claim 1 or 2, which is obtained by adding an N-terminal region of a Chlamydomonas reinhardtii-derived channelrhodopsin-1 to the N-terminus.

Further, a modified photoreceptive chloride channel described in claim 5 is the modified photoreceptive chloride channel described in claim 4, which is any of the following (a) to (c):
(a) a polypeptide composed of the amino acid sequence represented by SEQ ID NO: 5;
(b) a polypeptide that is composed of an amino acid sequence including deletion, substitution, addition, or insertion of one or a plurality of amino acids in the amino acid sequence represented by SEQ ID NO: 5, and that has a photoreceptive chloride channel function; and
(c) a polypeptide that is composed of an amino acid sequence having at least 90% sequence identity to the amino acid sequence represented by SEQ ID NO: 5, and that has a photoreceptive chloride channel function.

Further, a polynucleotide of the present invention, as described in claim 6, encodes the polypeptide described in any of claims 1 to 5.

Further, an expression vector of the present invention, as described in claim 7, includes the polynucleotide described in claim 6 functionally linked to a promoter.

Further, a cell of the present invention, as described in claim 8, expresses the polypeptide described in any of claims 1 to 5.

Further, a cell described in claim 9 is the cell described in claim 8, in which the cell is a cell that forms the retina.

Further, the present invention is directed to, as described in claim 10, use of any of the polypeptide described in any of claims 1 to 5, the polynucleotide described in claim 6, and the expression vector described in claim 7 in the production of a pharmaceutical for treating a subject suffering from damage to the outer retinal layers.

Further, use described in claim 11 is the use described in claim 10, in which the damage to the outer retinal layers is retinitis pigmentosa, age-related macular degeneration, or retinal detachment.

Further, a pharmaceutical composition for treating damage to the outer retinal layers of the present invention, as described in claim 12, contains either the polypeptide described in any of claims 1 to 5 or the expression vector described in claim 7 as an active ingredient.

### Effect of the Invention

According to the present invention, a modified photoreceptive chloride channel having excellent photoreactive properties in which the wavelength sensitivity range is narrow, and also both τon and τoff are short can be provided.

### BRIEF DESCRIPTION OF THE DRAWINGS

FIG. 1 A structure of the plasmid for preparing an adeno-associated virus vector expressing ChimGt12 in Example 1.

FIG. 2 A graph showing that ChimGt12 has a narrower wavelength sensitivity range than GtACR1 and GtACR2 in Test Example 1.

FIG. 3 A graph showing that ChimGt12 shows shorter τon than GtACR1 and GtACR2 in Test Example 1.

FIG. 4 A graph showing that ChimGt12 shows shorter τoff than GtACR1 and GtACR2 in Test Example 1.

FIG. 5 Graphs indicating that a decrease in retinal thickness can be significantly inhibited by introducing ChimGt12 gene into the retina in Test Example 3.

FIG. 6 Graphs indicating that the hyperpolarization response of photoreceptor cells is increased by introducing ChimGt12 gene into the retina in Test Example 3.

### MODE FOR CARRYING OUT THE INVENTION

A modified photoreceptive chloride channel of the present invention is a polypeptide obtained by substituting a region from the fourth transmembrane domain to the sixth transmembrane domain counted from the N-terminal side of GtACR1 isolated from Guillardia theta reported in Non-Patent Document 1 by a corresponding region of GtACR2. GtACR1 is a polypeptide (SEQ ID NO: 1) composed of the following 295 amino acids, and the region from the fourth transmembrane domain to the sixth transmembrane domain counted from the N-terminal side thereof is Asn123 to Phe213. GtACR2 is a polypeptide (SEQ ID NO: 2) composed of the following 291 amino acids, and the region from the fourth transmembrane domain to the sixth transmembrane domain counted from the N-terminal side thereof is Asn119 to Ile209 (for the amino acid sequences of GtACR1 and GtACR2, see, for example, Non-Patent Document 1 if needed).

```
(Amino acid sequence of GtACR1)
MSSITCDPAIYGEWSRENQFCVEKSLITLDGIKYVQLVMAVVSACQVFFMVTRAPKVPWEAIYLPTTEM
                                                          TM1 TM2
ITYSLAFTGNGYIRVANGKYLPWARMASWLCTCPIMLGLVSNMALVKYKSIPLNPMMIAASSICTVFGI
                                            TM3 TM4
TASVVLDPLHVWLYCFISSIFFIFEMVVAFAIFAITIHDFQTIGSPMSLKVVERLKLMRIVFYVSWMAY
              TM5 TM6
PILWSFSSTGACIMSENTSSVLYLLGDALCKNTYGILLWATTWGLLNGKWDRDYVKGRNVDGTLMPEYE
                              TM7
QDLEKGNTERYEDARAGET
※: transmembrane domain
(Amino acid sequence of GtACR2)
MASQVVYGEWASTHTECYNMSRIDSTFVSLLQLVWAVVSGCQTIFMISRAPKVPWESVYLPFVESITYA
                                                  TM1 TM2
LASTGNGTLQMRDGRFFPWSRWASWLCTCPIMLGQISNMALVKYKSIPLNPIAQAASIIRVVMGITATI
                                    TM3 TM4
SPAEYMKWLFFFFGATCLVFEYSVVFTIFQVGLYGFESVGTPLAQKVVVRIKMLRLIFFIAWTMFPIVW
      TM5 TM6
LISPTGVCVIHENVSAILYLLADGLCKNTYGVILWSTAWGVLEGKWDPACLPGQEKPEADDPFGLNHEK
                        TM7
NAPPNDEVNIRMFGR
※: transmembrane domain
```

The modified photoreceptive chloride channel of the present invention may be a polypeptide obtained by further modifying another domain or region in addition to having undergone substitution of a region from the fourth transmembrane domain to the sixth transmembrane domain counted from the N-terminal side of GtACR1 by a corresponding region of GtACR2. For example, it may be a polypeptide obtained by further substituting an intracellular domain between the third transmembrane domain and the fourth transmembrane domain counted from the N-terminal side of GtACR1, or an extracellular domain between the sixth transmembrane domain and the seventh transmembrane domain counted therefrom by a corresponding domain of GtACR2. The intracellular domain between the third transmembrane domain and the fourth transmembrane domain and the extracellular domain between the sixth transmembrane domain and the seventh transmembrane domain counted from the N-terminal side of each of GtACR1 and GtACR2 are composed of amino acids between TM3 and TM4 and amino acids between TM6 and TM7 described above, respectively.

As a specific example of the modified photoreceptive chloride channel of the present invention, a polypeptide composed of the amino acid sequence represented by SEQ ID NO: 3 is exemplified. This polypeptide (ChimGt12) is configured by substituting a region from the fourth transmembrane domain to the sixth transmembrane domain counted from the N-terminal side of GtACR1 by a corresponding region of GtACR2, and further substituting an extracellular domain between the sixth transmembrane domain and the seventh transmembrane domain counted therefrom by a corresponding domain of GtACR2.

Further, the modified photoreceptive chloride channel of the present invention may be a polypeptide obtained by adding an N-terminal region of a Chlamydomonas reinhardtii-derived channelrhodopsin-1, for example, all or some of the amino acids at positions 1 to 71 in the amino acid sequence of ChR1 represented by SEQ ID NO: 4 to the N-terminus. As a specific example thereof, a polypeptide composed of the amino acid sequence represented by SEQ ID NO: 5 is exemplified. This polypeptide (mV2Gt12) is configured by adding amino acids at positions 1 to 24 in the amino acid sequence of ChR1 represented by SEQ ID NO: 4 to the N-terminus of ChemGt12.

The modified photoreceptive chloride channel of the present invention includes a polypeptide that includes deletion, substitution, addition, or insertion of one or a plurality of amino acids in the amino acid sequence represented by each of SEQ ID NOS: 3 and 5, and that has a photoreceptive chloride channel function. Further, it includes a polypeptide that is composed of an amino acid sequence having at least 90% sequence identity to the amino acid sequence represented by each of SEQ ID NOS: 3 and 5, and that has a photoreceptive chloride channel function. Here, the “a plurality of” is an integer of 50 or less, preferably an integer of 30 or less, more preferably an integer of 10 or less, and, for example, 2 to 9, 2 to 7, or 2 to 5. The sequence identity to the amino acid sequence represented by each of SEQ ID NOS: 3 and 5 is preferably at least 91%, more preferably at least 92%, more preferably at least 938, more preferably at least 94%, more preferably at least 958, more preferably at least 96%, more preferably at least 978, more preferably at least 98%, and most preferably at least 99%. Note that the & of the identity refers to a value calculated using a software for calculating the identity between a plurality of (two) amino acid sequences (e.g., FASTA, DANASYS, BLAST, etc.) with default settings. Further, the “has a photoreceptive chloride channel function” means to have a channel function to control ion permeability between the outside and the inside of a cell by sensing light, which is well known to those skilled in the art, and it is preferred that at least one of the biological activities evaluated by the degree of light sensitivity, the light sensitive wavelength, the degree of ion permeability, τon, τoff, or the like is at least equivalent to the biological activity of the polypeptide composed of the amino acid sequence represented by each of SEQ ID NOS: 3 and 5.

The modified photoreceptive chloride channel of the present invention can be produced by a genetic engineering technique. Specifically, first, a polynucleotide encoding the modified photoreceptive chloride channel of the present invention (hereinafter, referred to as “the modified photoreceptive chloride channel gene of the present invention”) is prepared. The modified photoreceptive chloride channel gene of the present invention can be prepared by a method known to those skilled in the art. Specifically, for example, the gene can be prepared by chemical synthesis based on the sequence information of a polynucleotide encoding each of GtACR1 and GtACR2. Further, the gene can also be prepared by amplifying a desired region of each of the polynucleotides using PCR primers that amplify the desired region of each of the polynucleotides based on the sequence information of each of the polynucleotides, and linking these regions using, for example, Gibson Assembly System (New England Biolabs Ltd.) or the like. Subsequently, the modified photoreceptive chloride channel gene of the present invention functionally linked to a promoter is integrated into an expression vector that can maintain replication in a host bacterial cell, can stably express the encoded polypeptide, and can stably maintain the gene, a host is transformed using the obtained recombinant expression vector, and the modified photoreceptive chloride channel of the present invention can be produced in the host. For the recombination technique, Proc. Natl. Acad. Sci. USA., 1984 81: 5662, or Molecular Cloning: A Laboratory Manual (1989) Second Edition, Cold Spring Harbor Laboratory Press, or the like can be referred to. As the expression vector, Escherichia coli-derived plasmids (e.g., pET28, pGEX4T, pUC118, pUC119, pUC18, pUC19, and other plasmid DNAs), Bacillus subtilis-derived plasmids (e.g., pUB110, pTP5, and other plasmid DNAs), yeast-derived plasmids (e.g., YEp13, YEP24, YCp50, and other plasmid DNAs), A phages (Agt11 and AZAP), plasmids for use in mammals (pCMV and pSV40), virus vectors (e.g., animal virus vectors such as adenovirus vectors, adeno-associated virus vectors, retrovirus vectors, lentivirus vectors, or vaccinia virus vectors, and insect virus vectors such as baculovirus vectors), vectors for use in plants (e.g., binary vector pBI series), cosmid vectors, and the like can be used. Here, the “functionally linked” refers to a functional bond between a promoter sequence and a target polynucleotide sequence such that the promoter sequence can start transcription of the target polynucleotide sequence. The promoter is not particularly limited, and a suitable promoter may only be selected according to the host, and a known constitutive promoter or an inducible promoter can be used, but a constitutive promoter is preferably used. Specific examples thereof include CMV promoter, SV40 promoter, CAG promoter, synapsin promoter, rhodopsin promoter, CaMV promoter, glycolytic enzyme promoter, lac promoter, trp promoter, tac promoter, GAPDH promoter, GAL1 promoter, PH05 promoter, PGK promoter, thy1 promoter, GRK promoter, and RPEJ promoter. For the purpose of specifically expressing the modified photoreceptive chloride channel of the present invention in a specific cell, a transcriptional regulatory domain of a polypeptide gene specifically expressed in the cell (for example, a transcriptional regulatory domain of IRBP (Interphotoreceptor retinoid binding protein) which is specifically expressed in a photoreceptor cell (Marjorie Nicoud et al., The Journal of Gene Medicine, Volume 9, Issue 12, 1013-1107, December 2007)) may be linked upstream of such a promoter. The insertion of the modified photoreceptive chloride channel gene of the present invention into an expression vector is carried out, for example, by creating or linking a restriction enzyme site flanking the modified photoreceptive chloride channel gene of the present invention, and inserting the resultant into a restriction enzyme site or a multicloning site of a suitable vector DNA. The expression vector may include, in addition to a promoter and the modified photoreceptive chloride channel gene of the present invention, an enhancer and other cis elements, a splicing signal, a polyA addition signal, a selection marker (a drug resistance gene marker such as an ampicillin resistance marker ora tetracycline resistance marker, an auxotrophic complementary gene marker such as LEU1, TRP1, or URA3, a dominant selection marker such as APH, DHER, or TK, etc.), a ribosome binding site (RBS), or the like as needed. The transformation of the host can be carried out using a protoplast method, a spheroplast method, a competent cell method, a virus method, a calcium phosphate method, a lipofection method, a microinjection method, a gene bombardment method, an agrobacterium method, electroporation, or the like. The thus obtained transformant is cultured under appropriate conditions using a medium containing an assimilable carbon source, a nitrogen source, a metal salt, a vitamin, or the like. The culture of the transformant is usually carried out under aerobic conditions such as shake culture or aerated and agitated culture at 25 to 37° C. for 3 to 6 hours. The pH is kept around neutral during the period of culture. The pH is adjusted using an inorganic or organic acid, an alkaline solution, or the like. During the culture, an antibiotic such as ampicillin or tetracycline may be added to the medium according to the selection marker inserted into the recombinant expression vector as needed. Further, the host used for the transformation is not particularly limited as long as it can express the modified photoreceptive chloride channel of the present invention, and examples thereof include bacteria (Escherichia coli and Bacillus subtilis), yeasts (Saccharomyces cerevisiae, etc.), animal cells (COS cells, Chinese hamster ovary (CHO) cells, 3T3 cells, BHK cells, HEK 293 cells, etc.), and insect cells. The modified photoreceptive chloride channel of the present invention can be obtained in the form of retaining its activity by fractionation or purification using a common method from a culture (a culture supernatant, cultured cells, cultured bacterial cells, a homogenate of cells or bacterial cells, or the like) obtained by culturing the transformant, followed by ultrafiltration concentration, lyophilization, spray drying, crystallization, or the like. Alternatively, the modified photoreceptive chloride channel of the present invention may be provided in the form of cells expressing the modified photoreceptive chloride channel of the present invention without performing isolation or purification. In that case, the host cells used for the transformation are host cells suitable for subsequent use, for example, neurons (photoreceptor cells, bipolar cells, ganglion cells, etc.) that form the retina, or retinal pigment epithelial cells, preferably cells that form the human retina, but may be other cells. Further, when the modified photoreceptive chloride channel of the present invention is used for a medical application, it may be provided in the form of an expression vector for the modified photoreceptive chloride channel of the present invention. In that case, it is preferred to use an expression vector having excellent introduction efficiency into cells, replication maintenance in cells, stability, expression efficiency, and the like. Examples of such a vector can include virus vectors such as an adeno-associated virus vector, a retrovirus vector, and a lentivirus vector, (autonomously replicable) plasmids, and transposons. The plasmid for preparing an expression vector for the modified photoreceptive chloride channel of the present invention can be prepared according to the method described, for example, in Tomita H et al., Invest Ophthalmol Vis Sci. 2007 August; 48 (8): 3821-6, or Sugano E et al., Invest Ophthalmol Vis Sci. 2005 September; 46 (9): 3341-8.

Here, examples of the modified photoreceptive chloride channel gene of the present invention include a polynucleotide composed of the nucleotide sequence represented by SEQ ID NO: 6 (encoding a polypeptide composed of the amino acid sequence represented by SEQ ID NO: 3) and a polynucleotide composed of the nucleotide sequence represented by SEQ ID NO: 7 (encoding a polypeptide composed of the amino acid sequence represented by SEQ ID NO: 5). However, the modified photoreceptive chloride channel gene of the present invention is not limited to these polynucleotides, and includes a polynucleotide that hybridizes to a complementary strand of each of these polynucleotides under stringent conditions and encodes a polypeptide having a photoreceptive chloride channel function. Further, the gene includes a polynucleotide that has at least 90%, preferably at least 918, more preferably at least 92%, more preferably at least 93%, more preferably at least 94%, more preferably at least 95%, more preferably at least 96%, more preferably at least 978, more preferably at least 98%, and most preferably at least 998 sequence identity to the nucleotide sequence represented by each of SEQ ID NOS: 6 and 7 and that encodes a polypeptide having a photoreceptive chloride channel function. Here, the “hybridization under stringent conditions” includes, for example, hybridization in 3 to 4×SSC (150 mM sodium chloride, 15 mM sodium citrate, pH 7.2) and 0.1 to 0.5% SDS at 30 to 50° ° C. for 1 to 24 hours, preferably hybridization in 3.4×SSC and 0.3% SDS at 40 to 45° ° C. for 1 to 24 hours, and subsequent washing. As washing conditions, for example, conditions such as continuous washing with a solution containing 2×SSC and 0.1% SDS, a 1×SSC solution, and a 0.2×SSC solution at room temperature are exemplified. However, the combination of the above conditions is exemplary, and those skilled in the art can achieve the same stringency as described above by properly combining the above or other factors determining hybridization stringency (for example, the concentration, length, and GC content of a hybridization probe, the reaction time of hybridization, etc.).

The modified photoreceptive chloride channel of the present invention has excellent photoreactive properties in which the wavelength sensitivity range is narrower than that of GtACR1 and GtACR2, and also both τon and τoff are shorter than those of GtACR1 and GtACR2. The fact that the wavelength sensitivity range is narrow is effective in facilitating the designing of the selectivity of the wavelength range for controlling neuronal depolarization and inhibition, and the fact that both τon and τoff are short is effective in making it possible to control neurons with high time resolution. Therefore, the modified photoreceptive chloride channel of the present invention and the expression vector including a polynucleotide encoding the modified photoreceptive chloride channel are useful for treating a subject suffering from damage to the outer retinal layers by contributing to the inhibition of occurrence of visual dysfunction or visual function impairment due to the degeneration or loss of photoreceptor cells, the improvement of visual dysfunction or visual function impairment that has occurred, or the like. Here, the “damage to the outer retinal layers” refers to any disease in which cells other than photoreceptor cells remain normal or retain some of their functions although visual dysfunction or visual function impairment has occurred as by the degeneration or loss of photoreceptor cells present in the outer retinal layers. As such a disease, retinitis pigmentosa, age-related macular degeneration, retinal detachment, and the like can be exemplified. The “subject” means a subject with visual loss or a subject at risk for visual loss due to damage to the outer retinal layers. The subject is not limited to a human and may be any other mammal. Examples of such other mammal include mice, rats, monkeys, rabbits, dogs, cats, cattle, and horses. The “treatment of a subject suffering from damage to the outer retinal layers” means recovery of visual function as compared to before administration of the pharmaceutical of the present invention in a subject with visual loss or at risk for visual loss due to damage to the outer retinal layers. Further, the modified photoreceptive chloride channel of the present invention is also useful for various disorders associated with photoreaction such as disorders of the brain or the central/peripheral nervous system, spinal cord injury, and autoimmune diseases.

The pharmaceutical composition of the present invention contains the modified photoreceptive chloride channel of the present invention or the expression vector including a polynucleotide encoding the modified photoreceptive chloride channel as an active ingredient, and is formulated as a pharmaceutical for treating a subject suffering from damage to the outer retinal layers. The effective dose thereof is an amount that can have a therapeutic effect on a given symptom or usage, and is properly determined by those skilled in the art based on implementation of a test using an animal or a clinical test, however, the age, body weight, and sex of the subject being an administration target, the condition or severity of the disease, the administration method, and the like are considered. In the case of a virus, the viral dose is, for example, 1012 to 1013 capsids/ml (e.g., about 1013 capsids/ml). In the formulation as a pharmaceutical, the active ingredient may be formulated together with one or more pharmaceutically acceptable carriers. Examples of the pharmaceutically acceptable carrier include various buffer solutions, for example, saline and buffer solutions of phosphates, acetates, and the like. The pharmaceutical may contain another therapeutic ingredient. Examples of the another therapeutic ingredient include agents known as therapeutic agents for retinitis pigmentosa, age-related macular degeneration, retinal detachment, or the like. The pharmaceutical can be formulated, for example, into an injection for local administration, an eye drop, an eye wash, or the like. An injectable preparation can be provided, for example, as an ample or in a unit dosage form in a container for multiple administrations, by adding a preservative. Further, the pharmaceutical may be in the form of a lyophilized preparation to be reconstituted before use with a suitable vehicle, for example, pyrogen-free sterile water or the like. The pharmaceutical is preferably administered by direct injection into the affected area of a subject, that is, the retina, or direct contact with the vitreous body.

### EXAMPLES

Hereinafter, the present invention will be described in detail by way of Examples, however, the present invention should not be construed as being limited to the following description.

### Example 1: Modified Photoreceptive Chloride Channel of Present Invention Composed of Amino Acid Sequence Represented by SEQ ID NO: 3 (Acquisition of Cells Expressing ChimGt12)

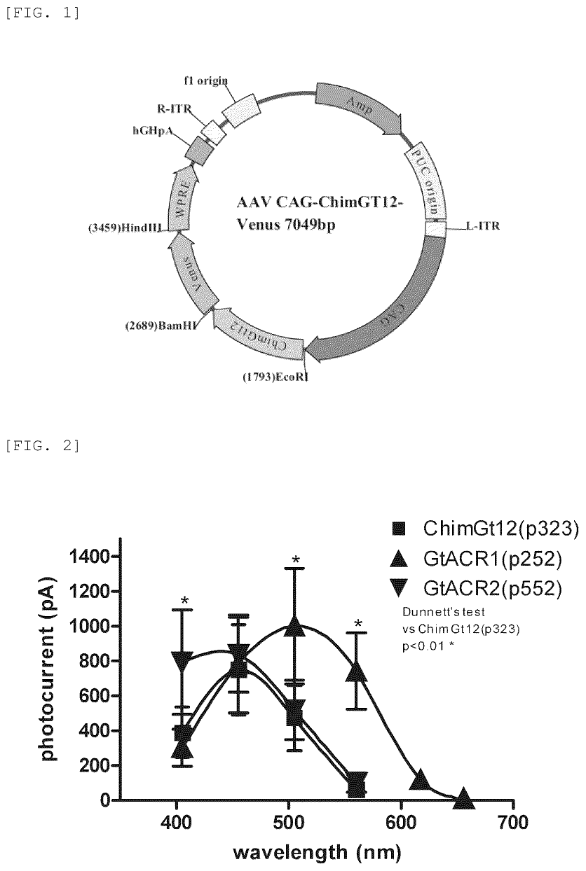

The cells expressing ChimGt12 were obtained in conformity to the method described in WO2011/019081 by the present inventors as follows. A polynucleotide, in which a polynucleotide encoding amino acids to the intracellular domain between the third transmembrane domain and the fourth transmembrane domain counted from the N-terminal side of GtACR1, a polynucleotide encoding amino acids from the fourth transmembrane domain counted from the N-terminal side of GtACR2 to the extracellular domain between the sixth transmembrane domain and the seventh transmembrane domain, and a polynucleotide encoding amino acids from the seventh transmembrane domain counted from the N-terminal side of GtACR1 to the C-terminus were linked, and a restriction enzyme sequence was added to the 5′ end and the 3′ end thereof, was chemically synthesized and inserted into a multicloning site of the plasmid for preparing an adeno-associated virus vector. The structure of the thus prepared plasmid for preparing an adeno-associated virus vector expressing ChimGt12 is shown in FIG. 1. In the plasmid, a fluorescent protein gene (venus) is located in a 3′ region of the multicloning site, and the target gene is expressed in the form of a fusion protein with venus attached to a C-terminal region. Therefore, this plasmid was transfected into cells by a calcium phosphate method, and a cell expressing ChimGt12 was specified using venus as an index. Specifically, to a tube containing a solution of the plasmid (the plasmid amount: 15 μg), 1.5 mL of 0.3 M CaCl₂) was added, followed by stirring by inversion, and then, the content was added to 1.5 mL of 2×HBS (280 mM NaCl, 1.5 mM Na₂HPO₄, 50 mM HEPES, pH 7.1) provided in another tube. The resultant was again stirred by inversion, and then added dropwise to HEK (Human Embryonic Kidney) 293 cells as a human embryonic kidney-derived cell line cultured in DMEM medium containing 10% FBS to transfect the plasmid, and the cells were cultured at 37° C. and 5% CO₂. After 6 hours, the medium was replaced with a fresh medium, and the cells were cultured for 2 days, and then, the cells were observed under a fluorescence microscope, thereby confirming the expression of ChimGt12 in the cells.

### Example 2: Modified Photoreceptive Chloride Channel of Present Invention Composed of Amino Acid Sequence Represented by SEQ ID NO: 5 (Acquisition of Cells Expressing mV2Gt12)

The cells expressing mV2Gt12 were prepared in the same manner as in Example 1 except that a polynucleotide, in which a polynucleotide encoding amino acids at positions 1 to 24 in the amino acid sequence of ChR1 represented by SEQ ID NO: 4 was linked to the 3′ end of the polynucleotide encoding the amino acids of ChimGt12, and a restriction enzyme sequence was added to the 5′ end and the 3′ end thereof, was chemically synthesized and inserted into a multicloning site of the plasmid for preparing an adeno-associated virus vector.

### Test Example 1: Measurement of Light-Induced Current and τon and τOff in Cells Expressing ChimGt12 by Patch Clamp Method

### (Measurement Method)

With respect to the cells expressing ChimGt12, after confirming the expression of venus under a microscope, measurement was performed using a patch clamp system (EPC-10, HEKA). As an extracellular solution, a solution composed of 138 mM NaCl, 3 mM KCl, 10 mM HEPES, 4 mM NaOH, 1 mM CaCl₂), and 2 mM MgCl₂ and adjusted to pH 7.4 with 1 N HCl was used. As a solution in the electrode, a solution composed of 130 mM CsCl, 1.1 mM EGTA, 2 mM MgCl₂, 0.1 mM CaCl₂), 10 mM NaCl, 10 mM HEPES, and 2 mM Na₂ATP and adjusted to pH 7.2 with 1 N CsOH was used. The light irradiation (light source: LED) was performed for 1 second, and the light intensity was set to 1 μW/mm², the stimulus duration was set to 60 seconds, and the holding potential was set to 0 mV. The wavelength was set to each of 405, 455, 505, 560, 617, and 656 nm.

### (Measurement Results)

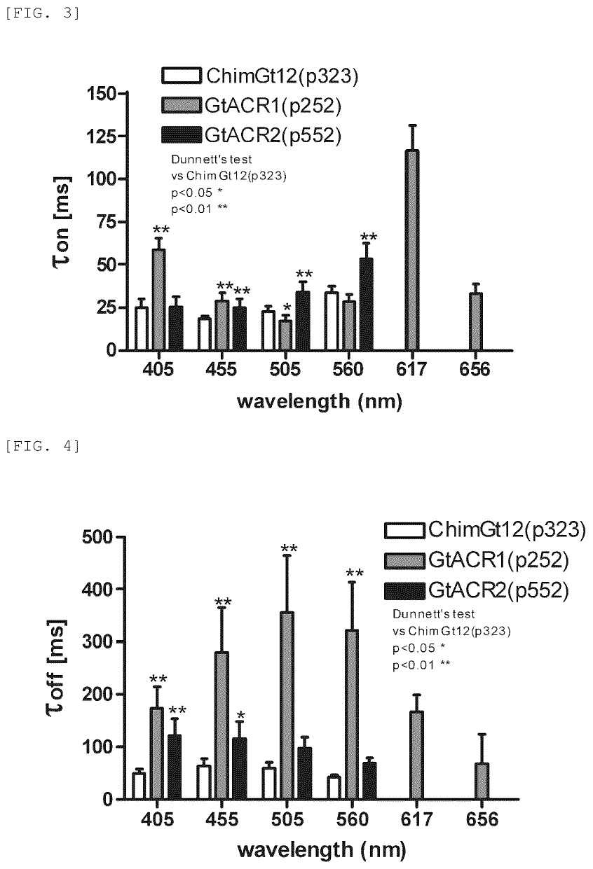

The measurement results of the light-induced current are shown in FIG. 2, and the measurement results of τon and τoff are shown in FIG. 3 and FIG. 4, respectively (n>11). In each figure, the measurement results for the cells expressing GtACR1 (n=7) and the cells expressing GtACR2 (n=8) obtained in the same manner as the cells expressing ChimGt12 are also shown. As apparent from FIG. 2, when the wavelength sensitivity range of ChimGt12 and the wavelength sensitivity range of GtACR1 were compared, both were equivalent on the short wavelength side, but on the long wavelength side, the wavelength sensitivity range of ChimGt12 was shorter than that of GtACR1. When the wavelength sensitivity range of ChimGt12 and the wavelength sensitivity range of GtACR2 were compared, both were equivalent on the long wavelength side, but on the short wavelength side, the wavelength sensitivity range of ChimGt12 was shorter than that of GtACR2. GtACR2 had high reactivity at 400 nm on the short wavelength side where the light energy is larger, and therefore, there was a concern about the occurrence of photodamage due to cell hyperpolarization. Further, as apparent from FIGS. 3 and 4, τon and τoff of ChimGt12 were shorter than those of GtACR1 and GtACR2 in the entire wavelength range with some exceptions at some wavelengths. When the fluorescent images of the cells expressing ChimGt12, the cells expressing GtACR1, and the cells expressing GtACR2 were compared, in the cells expressing GtACR1 and the cells expressing GtACR1, intense fluorescence emission considered to be caused by the fact that GtACR1 and GtACR2 do not maintain the proper conformation in the cells was observed, and therefore, there was a concern about the occurrence of cytotoxicity. However, such fluorescence emission was almost not observed in the cells expressing ChimGt12.

### Test Example 2: Measurement of Light-Induced Current and τon and τOff in Cells Expressing mV2Gt12 by Patch Clamp Method

The same measurement results as those of the cells expressing ChimGt12 were obtained by the same measurement method as in Test Example 1.

### Test Example 3: Introduction of ChimGt12 Gene into Retina Using Adeno-Associated Virus Vector and its Effect

### Experimental Method

### Preparation of Adeno-Associated Virus Vector

The adeno-associated virus vector for introducing the ChimGt12 gene into the retina was prepared from three types of plasmids: the plasmid for preparing an adeno-associated virus vector expressing ChimGt12, pAAV-RC, and pHelper using AAV Helper-Free System (Stratagene, La Jalla, CA) according to its manual. Specifically, to a tube after adding a solution of each plasmid (the amount of each plasmid was 15 μg) thereto and tapping, 1.5 mL of 0.3 M CaCl₂) was added, followed by stirring by inversion, and then, the content was added to 1.5 mL of 2×HBS (280 mM NaCl, 1.5 mM Na₂HPO₄, 50 mM HEPES, pH 7.1) provided in another tube. The resultant was again stirred by inversion, and then added dropwise to 293T cells cultured in a 15 cm culture dish to co-transfect the three types of plasmids by a calcium phosphate method, and the cells were cultured at 37° C. and 5% CO₂. After culturing for 3 days, the target virus particles were purified from the collected cells. Note that the plasmid for preparing an adeno-associated virus vector expressing ChimGt12 was prepared using GRK promoter, or RPEJ promoter in which for the purpose of specifically expressing ChimGt12 in a photoreceptor cell, a transcriptional regulatory domain of IRBP (Interphotoreceptor retinoid binding protein) which is specifically expressed in a photoreceptor cell (Marjorie Nicoud et al., The Journal of Gene Medicine, Volume 9, Issue 12, 1013-1107, December 2007) was linked upstream thereof, in place of CAG promoter used in the plasmid for preparing an adeno-associated virus vector expressing ChimGt12 prepared in Example 1. Further, as the control, the plasmid for preparing an adeno-associated virus vector for expressing only venus was prepared in the same manner as the plasmid for preparing an adeno-associated virus vector expressing ChimGt12 prepared in Example 1. As the serotype of the adeno-associated virus, M8 type (a mutated form in which Tyr at position 733 in a type 8 capsid protein was substituted by Phe according to Hilda Petrs-Silva et al., Molecular Therapy, Vol. 17, No. 3, 463-471, March 2009) or DJ type (Funakoshi Co., Ltd.) was used.

### Experimental Animal

16-Week-old or 24-Week-old P23H (line 2) rats were used. In P23H rats, the retina is once normally formed after birth, but the degeneration of photoreceptor cells proceeds gently, and about half of photoreceptor cells disappear 4 months after birth. After that, the degeneration of the photoreceptor cells still proceeds gently, and finally, almost all photoreceptor cells disappear, leading to visual loss.

Introduction of ChimGt12 Gene into Retina

Under mixed anesthesia of ketamine (66 mg/kg) and xylazine (3.3 mg/kg), the bulbar conjunctiva of both eyes of each P23H rat was incised about 1 mm, a 32-gauge microsyringe was inserted through the pars plana, and 5 μL of a virus solution was injected into the vitreous body. Alternatively, an upper part of the bulbar conjunctiva was incised, and the sclera was injured with a 30-gauge needle at a place about 1 mm away from the optic nerve, and 3 μL of a virus solution was subretinally administered with a 32-gauge microsyringe through the site.

### Measurement of Retinal Thickness

Before administration of the virus and every month after administration, the measurement was performed under mixed anesthesia of ketamine (66 mg/kg) and xylazine (3.3 mg/kg) in a state where the pupils were dilated with 18 atropine and 2.5% phenylephrine hydrochloride using a retinal optical coherence tomographer (OCT) (RS-3000 of NIDEK CO., LTD.).

### Measurement of Electroretinogram

Before administration of the virus and every month after administration, the electroretinogram was measured under mixed anesthesia of ketamine (66 mg/kg) and xylazine (3.3 mg/kg) in a state where the pupils were dilated with 1% atropine and 2.5% phenylephrine hydrochloride, and was recorded using an evoked response recorder (PuREC of Mayo Corporation). The light stimulation intensity was set in three stages of 0.01, 3.0, 10.0 cd·s/m².

### Experimental Results

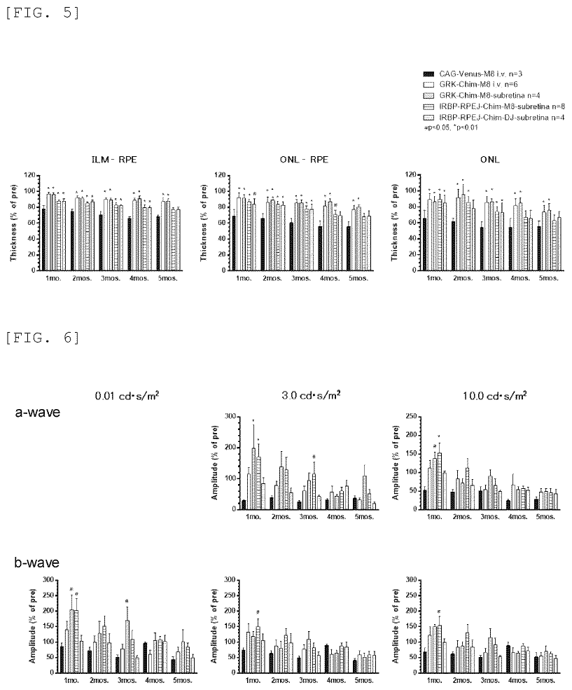

The measurement results of the retinal thickness are shown in FIG. 5. As apparent from FIG. 5, when the virus was intravitreally administered to express only venus (CAG-Venus-M8, i.v.), one month after the administration, the thicknesses of the all retinal layers (ILM-RPE), the photoreceptor layer (ONL-RPE), and the outer nuclear layer (ONL) decreased to about 75, about 70, and about 65, respectively, when the thickness before administration was taken as 100, and continued to decrease thereafter. On the other hand, when the virus was administered intravitreally (i.v.) or subretinally (subretina) to express ChimGt12, the decrease in thickness of each could be significantly inhibited. The measurement results of the electroretinogram are shown in FIG. 6. As apparent from FIG. 6, when the virus was intravitreally administered to express only venus, the hyperpolarization response of photoreceptor cells was significantly decreased compared to before the administration. On the other hand, when the virus was administered intravitreally or subretinally to express ChimGt12, one month after the administration, the hyperpolarization response of photoreceptor cells was increased compared to before the administration with some exceptions (in the case where the light stimulation intensity was 0.01 cd·s/m², the a-wave could not be measured). From the results, it was considered that ChimGt12 has an effect of not only inhibiting the degeneration of photoreceptor cells but also improving the function of photoreceptor cells.

### INDUSTRIAL APPLICABILITY

The present invention has industrial applicability in that it can provide a modified photoreceptive chloride channel having excellent photoreactive properties in which the wavelength sensitivity range is narrow, and also both τon and τoff are short.

1. A modified photoreceptive chloride channel, which is a polypeptide obtained by substituting a region from the fourth transmembrane domain to the sixth transmembrane domain counted from the N-terminal side of a Guillardia theta-derived photoreceptive chloride channel-1 (GtACR1) by a corresponding region of a Guillardia theta-derived photoreceptive chloride channel-2 (GtACR2).

2. The modified photoreceptive chloride channel according to claim 1, which is obtained by further substituting an intracellular domain between the third transmembrane domain and the fourth transmembrane domain counted from the N-terminal side of GtACR1, and/or an extracellular domain between the sixth transmembrane domain and the seventh transmembrane domain counted therefrom by a corresponding domain of GtACR2.

3. The modified photoreceptive chloride channel according to claim 1, which is any of the following (a) to (c):
(a) a polypeptide composed of the amino acid sequence represented by SEQ ID NO: 3;
(b) a polypeptide that is composed of an amino acid sequence including deletion, substitution, addition, or insertion of one or a plurality of amino acids in the amino acid sequence represented by SEQ ID NO: 3, and that has a photoreceptive chloride channel function; and
(c) a polypeptide that is composed of an amino acid sequence having at least 90% sequence identity to the amino acid sequence represented by SEQ ID NO: 3, and that has a photoreceptive chloride channel function.

4. The modified photoreceptive chloride channel according to claim 1, which is obtained by adding an N-terminal region of a Chlamydomonas reinhardtii-derived channelrhodopsin-1 to the N-terminus.

5. The modified photoreceptive chloride channel according to claim 4, which is any of the following (a) to (c):
(a) a polypeptide composed of the amino acid sequence represented by SEQ ID NO: 5;
(b) a polypeptide that is composed of an amino acid sequence including deletion, substitution, addition, or insertion of one or a plurality of amino acids in the amino acid sequence represented by SEQ ID NO: 5, and that has a photoreceptive chloride channel function; and
(c) a polypeptide that is composed of an amino acid sequence having at least 90% sequence identity to the amino acid sequence represented by SEQ ID NO: 5, and that has a photoreceptive chloride channel function.

6. A polynucleotide encoding the polypeptide according to claim 1.

7. An expression vector comprising the polynucleotide according to claim 6 functionally linked to a promoter.

8. A cell expressing the polypeptide according to claim 1.

9. The cell according to claim 8, wherein the cell is a cell that forms the retina.

10. Use of the polypeptide according to claim 1 in the production of a pharmaceutical for treating a subject suffering from damage to the outer retinal layers.

11. The use according to claim 10, wherein the damage to the outer retinal layers is retinitis pigmentosa, age-related macular degeneration, or retinal detachment.

12. A pharmaceutical composition for treating damage to the outer retinal layers, comprising the polypeptide according to claim 1 as an active ingredient.

13. The modified photoreceptive chloride channel according to claim 2, which is any of the following (a) to (c):
(a) a polypeptide composed of the amino acid sequence represented by SEQ ID NO: 3;
(b) a polypeptide that is composed of an amino acid sequence including deletion, substitution, addition, or insertion of one or a plurality of amino acids in the amino acid sequence represented by SEQ ID NO: 3, and that has a photoreceptive chloride channel function; and
(c) a polypeptide that is composed of an amino acid sequence having at least 90% sequence identity to the amino acid sequence represented by SEQ ID NO: 3, and that has a photoreceptive chloride channel function.

14. A polynucleotide encoding the polypeptide according to claim 2.

15. A polynucleotide encoding the polypeptide according to claim 3.

16. A polynucleotide encoding the polypeptide according to claim 4.

17. A polynucleotide encoding the polypeptide according to claim 5.

18. Use of the polynucleotide according to claim 6 in the production of a pharmaceutical for treating a subject suffering from damage to the outer retinal layers.

19. Use of the expression vector according to claim 7 in the production of a pharmaceutical for treating a subject suffering from damage to the outer retinal layers.

20. A pharmaceutical composition for treating damage to the outer retinal layers, comprising the expression vector according to claim 7 as an active ingredient.


SEQUENCE LISTING (7 sequences)

SEQ ID NO: 1 - protein, 295 aa, Guillardia theta
MSSITCDPAIYGEWSRENQFCVEKSLITLDGIKYVQLVMAVVSACQVFFMVTRAPKVPWEAIYLPTTEMITYSLAFTGNGYIRVANGKYLPWARMASWLCTCPIMLGLVSNMALVKYKSIPLNPMMIAASSICTVFGITASVVLDPLHVWLYCFISSIFFIFEMVVAFAIFAITIHDFQTIGSPMSLKVVERLKLMRIVFYVSWMAYPILWSFSSTGACIMSENTSSVLYLLGDALCKNTYGILLWATTWGLLNGKWDRDYVKGRNVDGTLMPEYEQDLEKGNTERYEDARAGET

SEQ ID NO: 2 - protein, 291 aa, Guillardia theta
MASQVVYGEWASTHTECYNMSRIDSTFVSLLQLVWAVVSGCQTIFMISRAPKVPWESVYLPFVESITYALASTGNGTLQMRDGRFFPWSRMASWLCTCPIMLGQISNMALVKYKSIPLNPIAQAASIIRVVMGITATISPAEYMKWLFFFFGATCLVFEYSVVFTIFQVGLYGFESVGTPLAQKVVVRIKMLRLIFFIAWTMFPIVWLISPTGVCVIHENVSAILYLLADGLCKNTYGVILWSTAWGVLEGKWDPACLPGQEKPEADDPFGLNHEKNAPPNDEVNIRMFGR

SEQ ID NO: 3 - protein, 295 aa, Artificial Sequence
  Fusion protein, ChimGt12
MSSITCDPAIYGEWSRENQFCVEKSLITLDGIKYVQLVMAVVSACQVFFMVTRAPKVPWEAIYLPTTEMITYSLAFTGNGYIRVANGKYLPWARMASWLCTCPIMLGLVSNMALVKYKSIPLNPIAQAASIIRVVMGITATISPAEYMKWLFFFFGATCLVFEYSVVFTIFQVGLYGFESVGTPLAQKVVVRIKMLRLIFFIAWTMFPIVWLISPTGVCVIHENTSSVLYLLGDALCKNTYGILLWATTWGLLNGKWDRDYVKGRNVDGTLMPEYEQDLEKGNTERYEDARAGET

SEQ ID NO: 4 - protein, 712 aa, Chlamydomonas reinhardtii
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGICRDLVRYLAWLYFCSWAMFPVLFLLGPEGFGHINQFNSAIAHAILDLASKNAWSMMGHFLRVKIHEHILLYGDIRKKQKVNVAGQEMEVETMVHEEDDETQKVPTAKYANRDSFIIMRDRLKEKGFETRASLDGDPNGDAEANAAAGGKPGMEMGKMTGMGMGMGAGMGMATIDSGRVILAVPDISMVDFFREQFARLPVPYELVPALGAENTLQLVQQAQSLGGCDFVLMHPEFLRDRSPTGLLPRLKMGGQRAAAFGWAAIGPMRDLIEGSGVDGWLEGPSFGAGINQQALVALINRMQQAKKMGMMGGMGMGMGGGMGMGMGMGMGMAPSMNAGMTGGMGGASMGGAVMGMGMGMQPMQQAMPAMSPMMTQQPSMMSQPSAMSAGGAMQAMGGVMPSPAPGGRVGTNPLFGSAPSPLSSQPGISPGMATPPAATAAPAAGGSEAEMLQQLMSEINRLKNELGE

SEQ ID NO: 5 - protein, 319 aa, Artificial Sequence
  Fusion protein, mV2Gt12
MSRRPWLLALALAVALAAGSAGASMSSITCDPAIYGEWSRENQFCVEKSLITLDGIKYVQLVMAVVSACQVFFMVTRAPKVPWEAIYLPTTEMITYSLAFTGNGYIRVANGKYLPWARMASWLCTCPIMLGLVSNMALVKYKSIPLNPIAQAASIIRVVMGITATISPAEYMKWLFFFFGATCLVFEYSVVFTIFQVGLYGFESVGTPLAQKVVVRIKMLRLIFFIAWTMFPIVWLISPTGVCVIHENTSSVLYLLGDALCKNTYGILLWATTWGLLNGKWDRDYVKGRNVDGTLMPEYEQDLEKGNTERYEDARAGET

SEQ ID NO: 6 - DNA, 885 bp, Artificial Sequence
  DNA encoding fusion protein, ChimGt12
atgagcagcatcacctgtgatcccgccatctacggcgagtggtcccgcgagaatcagttctgcgtggaaaagagcctgatcaccctggacggcatcaaatacgtgcagctggtcatggccgtggtgtccgcttgccaggtgttcttcatggtcacacgggcccctaaggtgccctgggaagctatctacctgcctaccaccgagatgatcacctacagcctggccttcaccggcaacggctatatcagagtggccaacggcaagtacctgccttgggccagaatggcctcctggctgtgtacatgccctatcatgctgggcctcgtgtccaacatggccctggtcaagtacaagagcatccctctgaaccctatcgctcaggccgccagcatcatcagagtcgtgatgggcatcaccgccaccatctctccagccgagtatatgaagtggctgttcttcttcttcggcgccacctgtctggtgttcgagtacagcgtggtgttcaccatcttccaagtgggcctgtacggcttcgagagcgtgggaacacctctggctcagaaagtggtcgtgcggatcaagatgctgcggctgatcttctttatcgcctggaccatgtttcccatcgtgtggctgattagccccaccggcgtgtgtgtgatccacgagaataccagcagcgtgctgtacctgctgggagatgccctgtgcaagaatacctacggcatcctgctgtgggccaccacatggggactgctgaatggcaagtgggacagagactacgtgaagggcagaaacgtggacggcaccctgatgcctgagtacgagcaggatctggaaaagggcaacaccgagagatacgaggatgccagagctggcgaaacg

SEQ ID NO: 7 - DNA, 957 bp, Artificial Sequence
  DNA encoding fusion protein, mV2Gt12
atgagcagaaggccttggctgctggctctggcactggctgttgctcttgctgctggatctgctggcgctagcatgagcagcatcacctgtgatcctgccatctacggcgagtggtcccgcgagaatcagttctgcgtggaaaagagcctgatcaccctggacggcatcaaatacgtgcagctggtcatggccgtggtgtccgcttgccaggtgttcttcatggttacacgggcccctaaggtgccctgggaagccatctatctgcccaccaccgagatgatcacctacagcctggccttcaccggcaacggctatatcagagtggccaacggcaagtacctgccttgggccagaatggcctcctggctgtgtacatgccctatcatgctgggcctcgtgtccaacatggccctggtcaagtacaagagcatccctctgaaccctatcgctcaggccgccagcatcatcagagtcgtgatgggcatcaccgccaccatctctccagccgagtatatgaagtggctgttcttcttcttcggcgccacctgtctggtgttcgagtacagcgtggtgttcaccatcttccaagtgggcctgtacggcttcgagagcgtgggaacacctctggctcagaaagtggtcgtgcggatcaagatgctgcggctgatcttctttatcgcctggaccatgtttcccatcgtgtggctgattagccccaccggcgtgtgtgtgatccacgagaataccagcagcgtgctgtacctgctgggagatgccctgtgcaagaatacctacggcatcctgctgtgggccaccacatggggactgctgaatggcaagtgggacagagactacgtgaagggcagaaacgtggacggcaccctgatgcctgagtacgagcaggatctggaaaagggcaacaccgagagatacgaggatgccagagctggcgaaacg